(19) United States
(12) Patent Application Publication
(10) Pub. No.: US 2003/0162292 A1
(43) Pub. Date: Aug. 28, 2003

(54) Method for producing heart-specific fluorescence of non-human eukaryotic animals

(75) Inventors: Huai-Jen Tsai, Taipei (TW); Chiu-Ju Huang, Taipei (TW); Chung-Der Hsiao, Taipei (TW)

Correspondence Address: BRUCE H. TROXELL, SUITE 1404, 5205 LEESBURG PIKE, FALLS CHURCH, VA, 22041, US

(73) Assignee: National Taiwan University

(21) Appl. No.: 10/079,528

(22) Filed: Feb. 22, 2002

(51) Int. Cl. A01K 67/00; C12N 15/85

(52) U.S. Cl. 435/455; 800/8

(57) ABSTRACT

A method of expressing in vivo heart-specific fluorescence in transgenic line of zebrafish is developed, which provides a research model for studying heart-related gene functions and performing gene therapies in the future. The method comprises the following step. A fluorescent protein gene is integrated into the genome of a non-human eukaryotic animal. In a preferred embodiment, a gene encoding GFP is transferred into the genome of a zebrafish. The transgenic process comprises the following steps. Firstly, the genomic DNA of zebrafish larvae are extracted and cut with a restriction enzyme. Then, the DNA fragments are ligated with adaptors, Pad1 and PR-SpeI. After ligation, PCR is performed twice to amplify the target DNA fragment. The amplified fragment is subjected to gene sequencing steps for determing the nucleotide sequence, which is the 5′ region of zebrafish cmlc2 gene. Subsequently, a plasmid is constructed. This plasmid construct includes the upstream regulatory region, the exon 1, the intron 1, and the exon 2 of cmlc2 gene, cDNA of GFP, wherein the cmlc2 gene and GFP cDNA form a cassette, and inverted terminal repeats from adeno-associated virus are flanked at both sides of this cassette. The plasmid construct is linearized and microinjected into one-celled zebrafish fertilized eggs. Lastly, the heart-specific fluorescent expressed zebrafish are selected and the germline-transmitting transgenic strain is generated.

### FIELD OF THE INVENTION

The present invention relates to a method of expressing in vivo heart-specific fluorescence and, more specifically, to a method of transferring a fluorescent protein gene into the genome of non-human eukaryotic animals, providing a research model for studying heart-related gene functions and performing heart-related drug and gene therapies in the future.

### BACKGROUND OF THE INVENTION

Heart disease is one of the most common causes of death in the world and the most troublesome symptoms. Therefore, a simple animal research model needs to be developed in order to make significant advances in finding a cure or novel gene for the heart.

Fish are the simplest vertebrates with heart organs. And, zebrafish (Denio rerio) has recently become a new experimental model for heart-related disease research. There are several advantages for using zebrafish to study heart-related diseases and genes:

(1) organogenesis can be easily observed because of the transparent embryos;

(2) shortened experiment time due to the rapid developmental processes;

(3) embryos can survive without a functional cardiovascular system, which makes cardiac defects be possible to analyze;

(4) the growth and pathological changes of zebrafish are observed more easily;

(5) heart contractile function of the zebrafish can be observed from its appearance, rendering sacrifices unnecessary;

(6) large-scale screen for mutants are possible and many cardiac morphogenesis and function mutants are available.

Despite these advantages, there has not been a research model zebrafish labeled with heart-specific fluorescence in vivo to date.

### SUMMARY OF THE INVENTION

The first objective of the present invention is to provide a method of expressing in vivo heart-specific fluorescence, serving as a research model, in non-human eukaryotic animals to trace the cell-fate of heart cells.

The second objective of the present invention is to provide a method of expressing in vivo fluorescence, serving as a research model, in non-human eukaryotic animals to search for new heart-specific genes.

The third objective of the present invention is to provide a method of expressing in vivo heart-specific fluorescence, serving as a research model, in non-human eukaryotic animals to serve as biological indices of environmental pollutants.

The fourth objective of the present invention is to provide a method of expressing in vivo heart-specific fluorescence, serving as a research model, in non-human eukaryotic animals to study the influences of new drugs applied to heart development and therapy.

A method of expressing in vivo heart-specific fluorescence in transgenic line of zebrafish is developed, which provides a research model for studying heart-related gene functions and performing drug and gene therapies in the future. The method comprises the following step. A fluorescent protein gene is integrated into the genome of a non-human eukaryotic animal. In a preferred embodiment, a gene encoding green fluorescent protein (GFP) is transferred into the genome of a zebrafish. The transgenic process comprises the following steps. First, the genomic DNA of zebrafish larvae are extracted and cut with a restriction enzyme at 37° C. Then, the DNA fragments are ligated with adaptors, Pad1 and PR-SpeI. After ligation, polymerase chain reaction (PCR) is performed twice to amplify the target DNA segment. The amplified segment is subjected to gene sequencing steps for determining the nucleotide sequence, which is the 5′ region of zebrafish cardiac myosin light chain 2 (cmlc2) gene. Subsequently, a plasmid is constructed. This plasmid includes the upstream regulatory region, the exon 1, the intron 1, and the exon 2 of cmlc2 gene, cDNA of GFP, wherein the cmlc2 gene fused with GFP cDNA form a cassette, and inverted terminal repeats from adeno-associated virus are flanked at both sides of this cassette. The plasmid construct is linearized with NotI digestion and subsequently microinjected into one-celled zebrafish fertilized eggs. Lastly, the heart-specific fluorescence expressed in embryos is screened under a fluorescence microscope. These putative founders mate with wild-type strains. A germ-line transmission of zebrafish possessing heart-specific fluorescence is developed.

### BRIEF DESCRIPTION OF THE DRAWINGS

The foregoing aspects and many of the attendant advantages of this invention will become more readily appreciated and understood by referencing the following detailed description in conjunction with the accompanying drawings, wherein:

FIG. 1 is a structural view of a plasmid construct, pICMLE, illustrating the composition of the construct in accordance with the present invention

FIG. 2 is a structural view of a linearized plasmid construct illustrating the segment compositions of the construct for gene transferring in accordance with the present invention

FIG. 3 shows the nucleotide sequence of the partial zebrafish cmlc2 gene; and

FIG. 4 shows the germ-line transmission of green-heart transgenic F2 derived from inter-crossing between two fluorescent F1 progeny in accordance with the present invention;

### DETAILED DESCRIPTION OF THE PREFERRED EMBODIMENT

A method is disclosed hereinafter to provide a method of expressing in vivo heart-specific fluorescence to provide a research model in non-human eukaryotic animals to trace the cell-fate of heart cells, to search for new heart-specific genes, to serve as biological indices of environmental pollutants and to study the influences of new drugs subjected to heart development and therapy. The method comprises transferring a fluorescent protein gene into the genome of non-human eukaryotic animals. This transgenic process includes the following steps. First, the genomic DNA of the non-human eukaryotic animals is extracted and cut with a restriction enzyme. Then, the DNA fragments are ligated with adaptors, Pad1 and PR-SpeI. PCR is performed twice to amplify the target DNA segment after ligation, and the amplified segment is subjected to gene sequencing steps. Continuously, a plasmid is constructed. This plasmid construct contains the partial heart-specific gene sequence of the non-human eukaryotic animals, cDNA of fluorescent protein, wherein the heart-specific gene fused with the fluorescent protein cDNA form a cassette, and inverted terminal repeats from adeno-associated virus are flanked at both sides of this cassette. The plasmid construct is linearized with Not1 digestion and subsequently microinjected into one-celled zebrafish fertilized eggs. Lastly, the heart-specific fluorescence expressed in embryos is screened under a fluorescence microscope. A stably germline-transmitting transgenic zebrafish possessing heart-specific fluorescence is generated. In a preferred embodiment, transferring a GFP gene into zebrafish is described much more in detailed as the followings. However, it will be appreciated that various changes can be made therein without departing from the spirit and scope of the invention to the person who skills in the art.

Genomic DNA Extraction and Restriction Enzyme Digestion

The genomic DNA was extracted from zebrafish larvae at 48 hour-post-fertilization. One μg of the extracted DNA was then added into 50 μl restriction enzyme buffer (50 mM NaCl, 10 mM Tris-HCl, 10 mM MgCl₂, 1 mM DTT, pH 7.9) and cut with SpeI restriction enzyme at 37° C. The DNA sample was purified by ethanol precipitation.

Adaptor Ligation

One μg of Spel-cut DNA fragment and 100 pmol Pad1 (TGCGAGTAAGGATCCTCACGCAAGGAATTCCGACCAGACACC) and PR-SpeI (P-CTAGGGTGTCTGGTCGC) adaptors were added in a final volume of 20 μl ligation buffer. The mixture was preheated in a GeneAmp PCR system at 70° C. for 20 minutes and then slowly cooled down to 4° C. (this took about 3 hours). After cooling, 6 units of AvrII restriction enzyme and 3 units of T4 DNA ligase (Promega) were added and reacted at 4° C. for 16 hours. Unligated adaptors were removed with Microcon-100 (Amicon) and the final volume of the sample was 50 μl. One μl DNA sample was used for the following PCR.

PCR and Its Product

Target DNA was amplified by performing PCR twice. The first PCR was carried out in a final volume of 20 μl solution containing 20 ng DNA (which had been ligated with adaptors served as a DNA template), 1 pmol of P1 primer (TGCGAGTAAGGATCCTCACGCA), 4 pmol of CML1 primer (ACTCCATCCCGGTTCTGATCT), 200 pmol of each dNTP, and 1 unit of VioTaq DNA polymerase (Viogene). Firstly, solution was heated at 94° C. for 1 minute to denature the DNA. Subsequently, the PCR was proceeded for 35 cycles. Each cycle was performed at 94° C. for 30 seconds and then at 68° C. for 6 minutes. Finally, this solution was treated at 68° C. for 8 minutes.

The first PCR product was then used to the second PCR. The second PCR was performed by using 1 μl of the first PCR product, 4 pmol P1 primer, 4 pmol CML2 primer (GGAGAAGACATTGGAAGAGCCT), and 1 unit of ExTaq (Takara). The second PCR products were identified by using agarose gel electrophoresis.

DNA Sequencing

The PCR product (1.6 kb) was purified from the agarose gel and inserted into pGEM-T vector for DNA sequencing. This PCR product was confirmed as the upstream regulatory region, exon 1, intron 1, and exon 2 of the zebrafish cmlc2 gene.

Plasmid Construct

Primers, CML4-XhoI (AACAACTCGAGTGTGACCAAAGCTTAAA TC) and CML2-NcoI (CTCAACCATGGAGAAGACATTGGAAGA), were designed based on the known sequences of above 1.6 kb PCR product in the pGEM-T. The uncut chromosomal DNA was served as a DNA template. The PCR was carried out in a 50 μl solution containing 100 ng DNA template, 10 pmol of each primer (CML4-XhoI and CML2-NcoI), 200 pmol of each dNTP, and 1 unit of ExTaq. Firstly, sample was heated at 94° C. for 1 minute to denature the DNA. Subsequently, the PCR was proceeded for 30 cycles. Each cycle was performed at 94° C. for 30 seconds, and then at 68° C. for 3 minutes. Lastly, the sample was treated at 68° C. for 8 minutes.

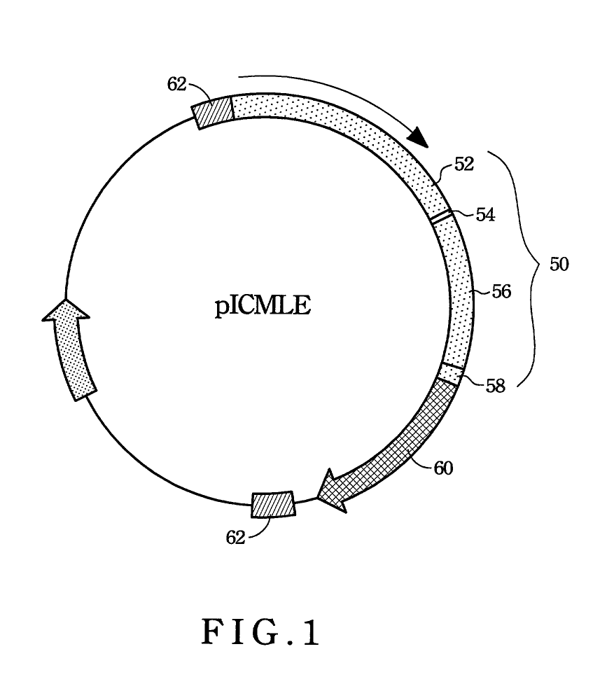

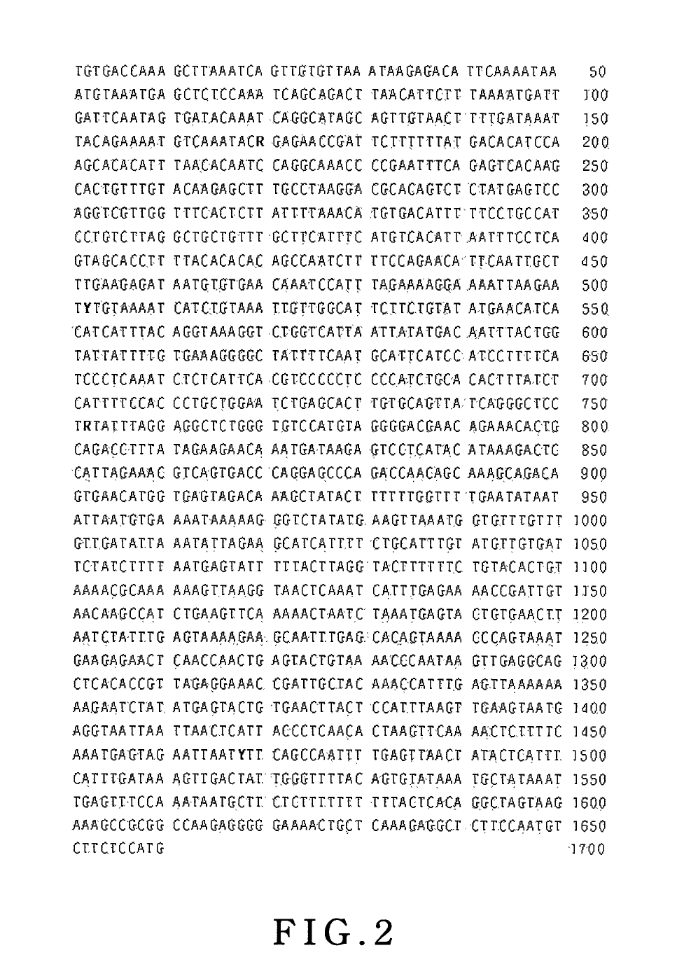

The final PCR product was cut with 20 units of the XhoI and NcoI in restriction enzyme buffer (50 mM NaCl, 10 mM Tris-HCl, 10 mM MgCl₂, 1 mM DTT, pH 7.9) at 37° C. for 20 hours. After that, the product was purified from the agarose gel and ligated into a plasmid pEGFP-ITR, which was cut by XhoI and NcoI. The resultant plasmid construct was designated as pICMLE (FIG. 1). The construct comprised cmlc2 gene 50 of the zebrafish. And the nucleotide sequence of the cmlc2 gene 50 illustrated in FIG. 2 includes 869 bp of upstream regulatory region 52, 40 bp of exon 1 54, 682 bp of intron 1 56 and 69 bp of exon 2 58. This 1.6 kb segment was then fused with GFP cDNA 60 to form a cassette. And this cassette was flanked by 145 bp of inverted terminal repeats 62 derived from adeno-associated virus at both sides. The GFP expression was controlled by cmlc2 regulatory region of the zebrafish.

Zebrafish Breeding

A pool of male and female zebrafish were kept in a 60×20×30 cm glass aquarium set to 28.5° C. and a 14 hour photoperiod. The fish were fed with artemia twice a day. After that, several pairs of strong male and female zebrafish were selected and put in the 30×10 ×20 cm breeding cage equipped with a net for collecting eggs. For transgenic line, one pair of transgenic individual and wild-type were kept in a 22×14×13 cm tank.

Gene Transferring and Transgenic Founders' Screening

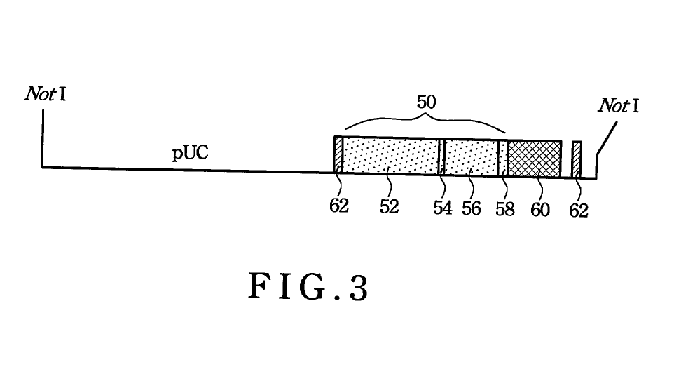

The fertilized eggs were collected with a plastic capillary and placed in a holder. A glass needle with 10 μm opening was filled with the NotI-cut plasmid pICMLE (FIG. 3) solution and mounted with mineral oil. The DNA sample was then microinjected into the one-celled fertilized eggs in a volume of 2-4 nl.

The injected fertilized eggs were incubated in dishes containing low concentration of methylene blue solution and placed in an incubator set to 28° C. Heart development and green fluorescent expression of the embryos were observed in the third day by using a fluorescence microscope.

After injecting the construct into the fertilized eggs for three days, a 50 to 70% survival rate of the transferred zebrafish embryos was obtained. There were 45 to 50% survival embryos having green fluorescent expression. Five days later, the heart-specific fluorescent zebrafish were moved to an aquarium for rearing. Sexual maturation was achieved after 12 weeks.

Generation of Germ-Line Transmission of cmlc2-GFP Transgenic Zebrafish

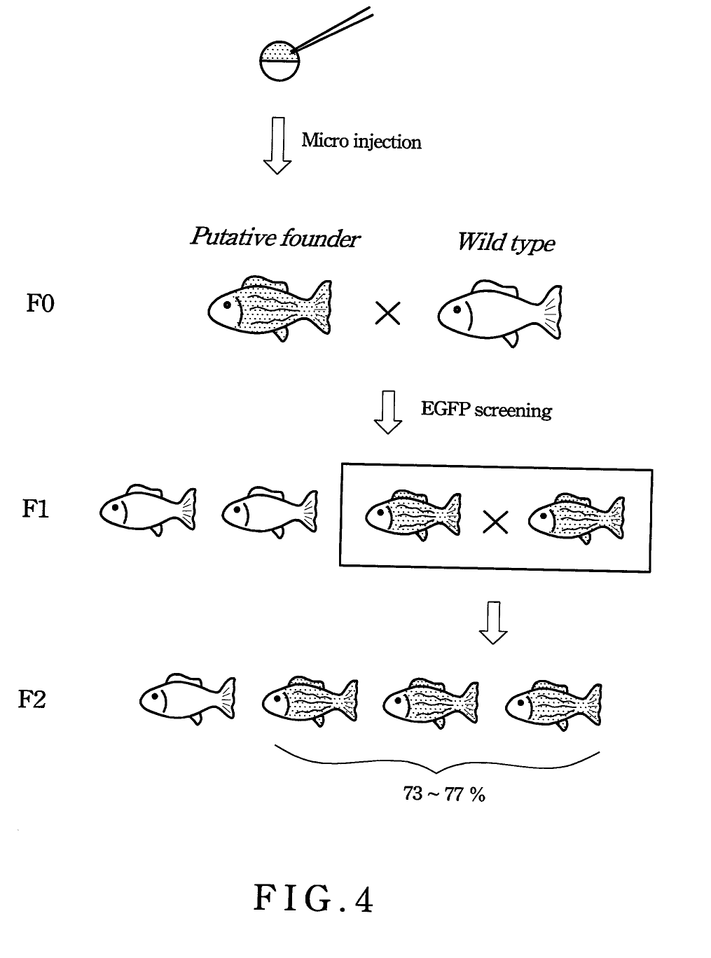

To generate germ-line transmitting transgenic zebrafish, the linearized pICMLE was injected into zebrafish eggs at one-celled stage. Approximately 50% of injected embryos expressed GFP in heart were raised to adult stage. The putative founders with the GFP expression were crossing with the wild-type strains. Among 324 founders, 37 individuals (11.4%) produced GFP-expressing offsprings, in which 34 lines showed heart-specific fluorescence. The transgenic transmission rates of F2 (the second progeny) derived from inter-crossing between two fluorescent F1 were 73 to 77% (FIG. 4), which were all following the Mendelian inheritance rule, indicating that the transgene in these transgenic lines was integrated into a single chromosomal locus. The heart-specific fluorescent expression could still be observed in the fish over their entire lifespan.

While the preferred embodiment of the invention has been illustrated and described, it is appreciated that various changes can be made therein without departing from the spirit and scope of the invention.

What is claimed:

1. A method of expressing in vivo heart-specific fluorescence of non-human eukaryotic animals, comprising of the following steps:
transferring a gene fragment encoding a fluorescent protein into genome of said non-human eukaryotic animals for expressing said in vivo heart-specific fluorescence.

2. The method of claim 1, wherein said non-human eukaryotic animals can be chosen from zebrafish.

3. The method of claim 1, wherein said fluorescent protein gene can be selected from GFP gene and said fluorescent expression is regulated by cmlc2 gene of said non-human eukaryotic animals.

4. The method of claim 1, wherein said gene transferring further comprises the following steps:
extracting said chromosomal DNA of said non-human eukaryotic animals;
cutting said chromosomal DNA with restriction enzymes to form DNA fragments;
ligating said DNA fragments with adaptors;
performing gene sequencing of said DNA fragments; and
engineering plasmid construct, wherein said plasmid construct comprises said fluorescent protein gene and said cmlc2 gene.

5. The method of claim 4, wherein said restriction enzymes can be chosen from SpeI.

6. The method of claim 4, wherein said adaptors can be selected from Pad1, PR-SpeI or any combination thereof.

7. The method of claim 6, wherein the sequences of said Pad1 and said PR-SpeI are TGCGAGTAAGGATCCTCACGCAAGGAATTCCGAC CAGACACC and P-CTAGGGTGTCTGGTCGC, respectively.

8. The method of claim 4, wherein said cmlc2 gene and said fluorescent protein gene form a cassette, and said plasmid construct further comprises inverted terminal repeats of adeno-associated virus flanked at both sides of said cassette.

9. A method of expressing in vivo heart-specific fluorescence in non-human eukaryotic animals, comprising of the following steps:
transferring a gene fragment encoding a fluorescent protein into genome of said non-human eukaryotic animals for expressing said in vivo heart-specific fluorescence, wherein said in vivo heart-specific fluorescent expression is regulated by a cmlc2 gene of said non-human eukaryotic animals.

10. The method of claim 9, wherein said non-human eukaryotic animals can be chosen from zebrafish.

11. The method of claim 9, wherein said fluorescent protein gene can be selected from GFP gene.

12. The method of claim 9, wherein said gene transferring further comprises the following steps:
extracting said chromosomal DNA of said non-human eukaryotic animals;
cutting said chromosomal DNA with restriction enzymes to form DNA fragments;
ligating said DNA fragments with adaptors;
performing gene sequencing of said DNA fragments; and
engineering plasmid construct, wherein said plasmid construct comprises said fluorescent protein gene and said cmlc2 gene.

13. The method of claim 12, wherein said restriction enzymes can be chosen from SpeI.

14. The method of claim 12, wherein said adaptors can be selected from Pad1, PR-SpeI or the combination thereof.

15. The method of claim 12, wherein said cmlc2 gene and said fluorescent protein gene form a cassette, and said plasmid construct further comprises inverted terminal repeats of adeno-associated virus flanked at both sides of said cassette.

16. A method of expressing in vivo heart-specific fluorescence in non-human eukaryotic animals, comprising of the following steps:
engineering a plasmid construct comprising a fluorescent protein gene and a cmlc2 gene, wherein said cmlc2 gene is derived from said non-human eukaryotic animals; and
integrating said plasmid construct into genome of said non-human eukaryotic animals for expressing said in vivo heart-specific fluorescence.

17. The method of claim 16, wherein said non-human eukaryotic animals can be chosen from zebrafish.

18. The method of claim 16, wherein said fluorescent protein gene can be selected from GFP gene and said fluorescent expression is regulated by said cmlc2 gene of said non-human eukaryotic animals.

19. The method of claim 16, wherein said cmlc2 gene and said fluorescent protein gene form a cassette, and said plasmid construct further comprises inverted terminal repeats of adeno-associated virus flanked at both sides of said cassette.

20. A heart-specific fluorescent fish is derived from transferring a gene fragment encoding a fluorescent protein into genome of said fish, wherein said heart-specific fluorescent expression is regulated by cmlc2 gene of said fish.
